(19) United States
(12) Patent Application Publication
(10) Pub. No.: US 2005/0112585 A1
(43) Pub. Date: May 26, 2005

(54) Method for adjusting the quantification range of individual analytes in a multiplexed assay

(76) Inventors: Dominic Zichi, Boulder, CO (US); Larry Gold, Boulder, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/719,894

(22) Filed: Nov. 21, 2003

(51) Int. Cl. C12Q 1/68

(52) U.S. Cl. 435/6

(57) ABSTRACT

The invention provides general methods for adjusting the inherent quantification range of a particular set of analytes in assays that employ capture reagents immobilized on solid supports. Specifically, the quantification range of a given analyte is adjusted to higher concentration regions by the addition of free capture reagent specific for that analyte, leaving the range of the remaining analytes the same and thereby permitting the simultaneous and accurate quantification of a plurality of analytes over a wide range of concentration values.

### FIELD OF THE INVENTION

The invention is directed towards multiplexed assays for analytes. Specifically, the invention is directed towards methods and reagents for simultaneously quantifying high and low abundance analytes that may be contained within a biological fluid.

### BACKGROUND OF THE INVENTION

The ability to quantify multiple analyte levels in biological fluids or extracts promises to revolutionize biological and medical research. In particular, measurements of the levels of protein analytes in an organism, termed the proteome, are key to understanding the current state of the organism and will change as the state changes; the diagnostic potential of such information is widely appreciated. Such proteomic measurements are the direct analogue of genomic measurements made with DNA microarrays with several important differences. Gene expression arrays typically quantify the levels of mRNA in a sample and these levels do not always correlate well with protein levels. Further, no information regarding post-translation modification of proteins can be extracted from gene expression data, whereas capture reagents, such as nucleic acid ligands or antibodies, can be made to discriminate between different protein modifications. Finally, the physiological range of protein levels in an organism vary over a wider range, at least 10 logs, than mRNA levels, which encompass ˜4-5 logs. For example, cytokines typically occur at subfemtomolar concentrations while many complement proteins approach micromolar concentrations.

The wide range of physiologic analyte levels poses a challenging problem to the multiplexed measurements of analytes within a single experiment. To date, protein levels have been measured individually with assays tailored to each analyte of interest. Low level analytes may be detected with signal amplification schemes and high abundant analytes may simply be diluted in order to bring physiologic levels into the optimal quantification range of the assay. Obviously, no such general solution can exist for proteomics measurements since it is necessary to measure both high and low abundant proteins simultaneously. In principle, high abundant analytes could be measured with capture reagents with affinities, quantified by the dissociation constant K_{d}, comparable to their physiologic levels. This becomes problematic from a specificity standpoint, since weaker specific interactions compete with a variety of weak nonspecific ones. Such nonspecific interactions are primarily responsible for background effects and therefore set the lower limit of detection. Also, when multiplexing assays, the protocols must be adjusted to accommodate the poorest performing ones; weaker interactions most likely have short off rates compared with high affinity ones and therefore can limit the effectiveness of washing background away, for example.

Clearly, it is desirable to use high affinity, high specificity capture reagents—including, but not limited to, antibodies and nucleic acid ligands—in a microarray setting. With uniformly high affinity capture reagents, the lower limit of detection is generally comparable among analytes; it is the upper limit of quantification that is difficult to tailor to each analyte within a multiplexed assay. For assays that employ high affinity interactions, it is the overall concentration of the capture reagent that sets the upper limit of quantification in a sample. The concentration of individual capture reagents in a microtiter plate, or on beads, etc., is limited to nanomolar concentrations at best and is more typically in the 10-100 picomolar range for microarrays. The detection of low level analytes limits sample dilution to ˜10%; it becomes difficult to simultaneously measure higher abundant analytes with endogenous levels exceeding nM.

The object of the current invention is to provide a general method for adjusting the inherent quantification range of a particular set of analytes to higher concentration regions, leaving the range of the remaining analytes the same and thereby permitting the simultaneous and accurate quantification of a plurality of analytes over a wide range of concentration values.

### SUMMARY OF THE INVENTION

The invention includes a method for decreasing the amount of a first analyte in a biological fluid that is capable of binding to a solid support-immobilized first capture reagent without decreasing the amount of a second analyte in the same biological fluid that is capable of binding to a solid support-immobilized second capture reagent. The method involves contacting the biological fluid with a quantity of the first capture reagent free in solution. The addition of a quantity of the first capture reagent free in solution quantitatively specifically titrates the amount of the first analyte captured in the assay, lowering saturating levels of the first analyte to quantifiable levels.

In embodiments in which the dissociation constant, K_{d}, of the first analyte for the first capture reagent is greater than the concentration, Cₛ, of the first capture reagent immobilized on the solid support, the concentration of the first capture reagent free in solution is preferably greater than said dissociation constant, more preferably 10 fold greater.

In embodiments in which the dissociation constant, K_{d}, of the first analyte for the first capture reagent is less than the concentration, Cₛ, of said first capture reagent immobilized on said solid support, the concentration of the first capture reagent free in solution is preferably greater than Cₛ, more preferably 10 fold greater.

The methods may be applied to multiplexed assays in which thousands of analytes must be assayed simultaneously in a biological fluid. For each abundant analyte, a quantity of cognate capture reagent may be added to the biological fluid in order to shift the concentrations of those abundant analytes to quantifiable levels while retaining the sensitivity desired for low abundance analytes.

The invention also provide a method for determining the concentration of an analyte in a biological fluid. The method involves providing a solid support upon which is immobilized a first quantity of a capture reagent that is capable of binding to the analyte in the biological fluid. The solid support is then contacted with a mixture comprising the biological fluid to be assayed and a second quantity of the capture reagent. The amount of analyte bound to the solid support is then measured. The concentration of the analyte in the biological fluid may then be determined based on the measurement of the amount of the analyte that has bound to the solid support, the concentration of the second quantity of the capture reagent in the mixture, and the K_{d} of said capture reagent.

The invention also provides a method for lowering the nonspecific binding of an analyte in a biological fluid to a non-cognate capture reagent immobilized on a solid support. The method involves contacting the biological fluid with free capture reagent capable of specifically binding to the analyte.

### BRIEF DESCRIPTION OF THE DRAWINGS

FIG. 1 depicts a standard curve shift using a multiplexed aptamer microarray. The left most curve (circles) is the standard curve in buffer for angiogenin protein using aptamer 1069-1 with no soluble aptamer, while the two left shifted curves were generated using 1 nM (squares) and 10 nM (triangles) soluble 1069-1 in the assay diluent.

FIG. 2 depicts a standard curve shift for angiogenin using a multiplexed aptamer microarray and seven soluble aptamers at varying concentrations. The left most curve (circles) is the standard curve in buffer and the right most curve (squares) is the standard curve with soluble aptamer in the assay diluent. The standard curve data points are displayed as filled markers and seven serum measurements are displayed as open markers on the two standard curves.

FIG. 3 depicts a standard curve shift for endostatin using a multiplexed aptamer microarray and seven soluble aptamers at varying concentrations. The left most curve (circles) is the standard curve in buffer and the right most curve (squares) is the standard curve with soluble aptamer in the assay diluent. The standard curve data points are displayed as filled markers and seven serum measurements are displayed as open markers on the two standard curves.

FIG. 4 depicts a standard curve shift for IgE using a multiplexed aptamer microarray and seven soluble aptamers at varying concentrations. The left most curve (circles) is the standard curve in buffer and the right most curve (squares) is the standard curve with soluble aptamer in the assay diluent. The standard curve data points are displayed as filled markers and seven serum measurements are displayed as open markers on the two standard curves.

FIG. 5 depicts a standard curve shift for P-selectin using a multiplexed aptamer microarray and seven soluble aptamers at varying concentrations. The left most curve (circles) is the standard curve in buffer and the right most curve (squares) is the standard curve with soluble aptamer in the assay diluent. The standard curve data points are displayed as filled markers and seven serum measurements are displayed as open markers on the two standard curves.

FIG. 6 depicts a standard curve shift for TIMP-1 using a multiplexed aptamer microarray and seven soluble aptamers at varying concentrations. The left most curve (circles) is the standard curve in buffer and the right most curve (squares) is the standard curve with soluble aptamer in the assay diluent. The standard curve data points are displayed as filled markers and seven serum measurements are displayed as open markers on the two standard curves.

FIG. 7 depicts a standard curve shift for lactoferrin using a multiplexed aptamer microarray and seven soluble aptamers at varying concentrations. The left most curve (circles) is the standard curve in buffer and the right most curve (squares) is the standard curve with soluble aptamer in the assay diluent. The standard curve data points are displayed as filled markers and seven serum measurements are displayed as open markers on the two standard curves.

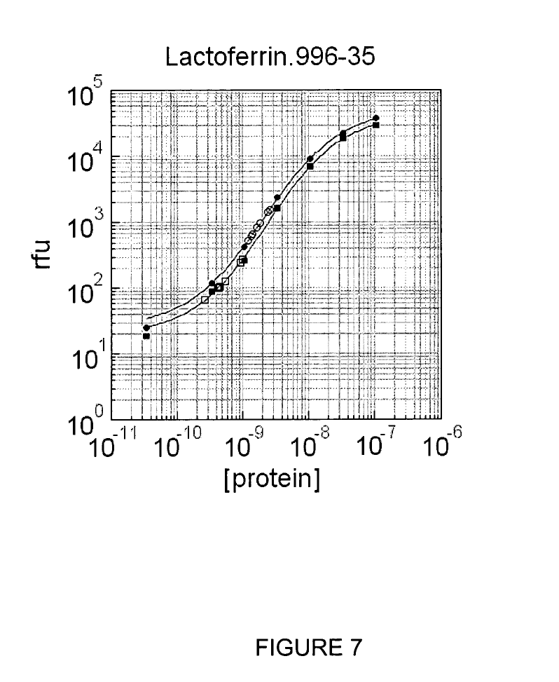

FIG. 8 depicts a standard curve shift for L-selectin using a multiplexed aptamer microarray and seven soluble aptamers at varying concentrations. The left most curve (circles) is the standard curve in buffer and the right most curve (squares) is the standard curve with soluble aptamer in the assay diluent. The standard curve data points are displayed as filled markers and seven serum measurements are displayed as open markers on the two standard curves.

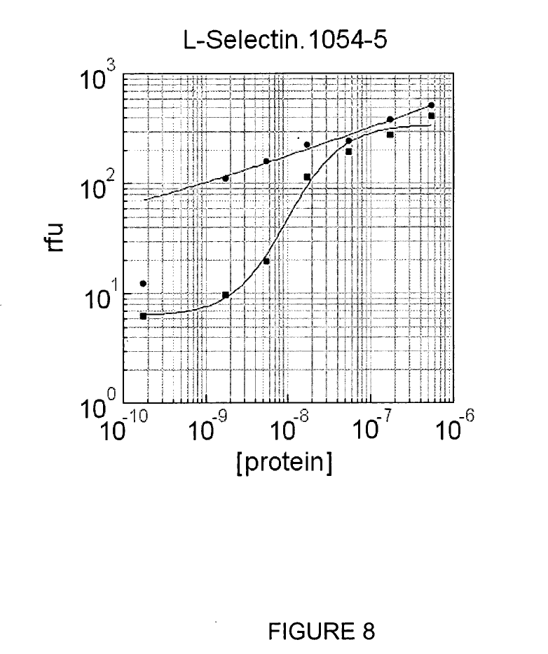

FIG. 9 depicts six serum sample spikes for IgE. The serum spikes (upper most curves) are seen to converge to the standard curve in buffer (filled circles) with no evidence of differential matrix effects.

FIG. 10 illustrates that the computed concentrations in FIG. 9 are in excellent agreement with the spiked values, even for the lowest levels spiked within large endogenous levels.

FIG. 11 depicts six serum sample spikes for TIMP-1. The serum spikes (upper most curves) are seen to converge to the standard curve in buffer (filled circles) with no evidence of differential matrix effects.

FIG. 12 illustrates that the computed concentrations in FIG. 11 are in excellent agreement with the spiked values, even for the lowest levels spiked within large endogenous levels.

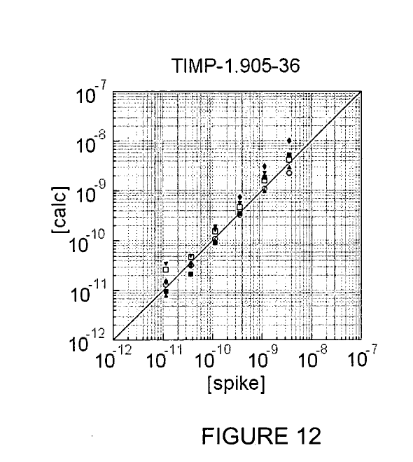

FIG. 13 depicts in boxplot format the coefficients of variation for all aptamers in a microarray using seven individual soluble aptamers. The data are presented as boxplots where 50% of the measurements lie within the boxes, the median is denoted by the white bar through the box and the lines above and below the box indicate the data range.

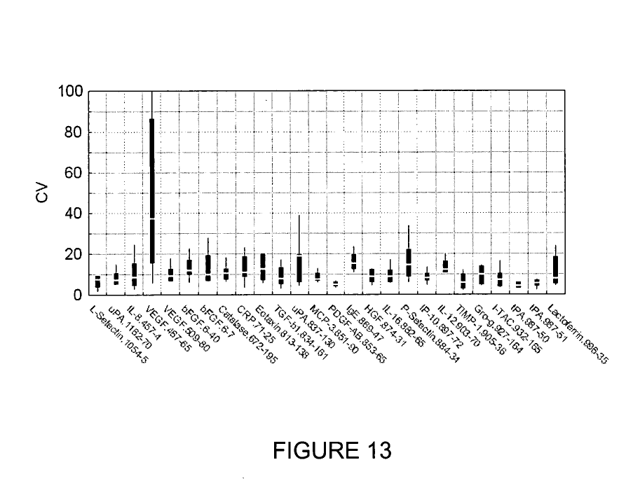

### DETAILED DESCRIPTION OF THE PREFERRED EMBODIMENTS

Definitions

Various terms are used herein to refer to aspects of the present invention. To aid in the clarification of the description of the components of this invention, the following definitions are provided:

The term “capture reagent” means a molecule or a multi-molecular complex that can bind to an analyte. Capture agents preferably bind their analyte binding partners in a substantially specific manner. The capture reagent may optionally be a naturally occurring, recombinant, or synthetic biomolecule. Antibodies or antibody fragments and nucleic acid ligands (aptamers) are highly suitable as capture agents. Antigens may also serve as capture agents for protein analytes, since they are capable of binding antibodies. A receptor that binds a protein ligand is another example of a possible capture reagent. Capture agents are understood not to be limited to agents that only interact with their analyte binding partners through noncovalent interactions. Capture agents may also optionally become covalently attached to the analytes which they bind. For instance, the capture reagent may be a photocrosslinking nucleic acid ligand that becomes photocrosslinked to its analyte binding partner following binding and photo activation.

The term “cognate” is sometimes used to indicate that a particular analyte binds in a substantially specific manner to a particular capture reagent i.e., an analyte binds in a substantially specific manner to its cognate capture reagent, but may bind in a non-specific manner to other noncognate capture reagents (which noncognate capture reagents in turn bind in a substantially specific manner to other analytes).

As used herein, the term “analyte” refers to any compound to be detected in an assay via its binding to a capture reagent. An analyte can be a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, etc., without limitation.

As used herein, the term “biological fluid” refers to a mixture of macromolecules obtained from an organism. This includes, but is not limited to, blood plasma, urine, semen, saliva, lymph fluid, meningial fluid, amniotic fluid, glandular fluid, and cerebrospinal fluid. This also includes experimentally separated fractions of all of the preceding. The term “biological fluid” also includes solutions or mixtures containing homogenized solid material, such as feces, tissues, and biopsy samples.

As used herein, “solid support” is defined as any surface to which molecules may be attached through either covalent or non-covalent bonds. This includes, but is not limited to, membranes, plastics, paramagnetic beads, charged paper, nylon, Langmuir-Bodgett films, functionalized glass, germanium, silicon, PTFE, polystyrene, gallium arsenide, gold and silver. Any other material known in the art that is capable of having functional groups such as amino, carboxyl, thiol or hydroxyl incorporated on its surface, is also contemplated. This includes surfaces with any topology, including, but not limited to, spherical surfaces, grooved surfaces, and cylindrical surfaces e.g., columns. Multiple capture reagents, each specific for a different analyte, may be attached to specific locations (“addresses”) on the surface of a solid support in an addressable format to form an array, also referred to as a “microarray” or as a “biochip.” By way of non-limiting example only, an array may be formed with a planar solid support, the surface of which is attached to capture reagents. By way of non-limiting example only, an array may also be formed by attaching capture reagents to beads, followed by placing the beads in an array format on another solid support, such as a microtiter plate.

As used herein, “nucleic acid ligand” is a non-naturally occurring nucleic acid having a desirable action on a target. Nucleic acid ligands are also referred to in this application as “aptamers.” A desirable action includes, but is not limited to, binding of the target, catalytically changing the target, reacting with the target in a way that modifies/alters the target or the functional activity of the target, covalently attaching to the target as in a suicide inhibitor, facilitating the reaction between the target and another molecule. In the preferred embodiment, the action is specific binding affinity for a target molecule, such target molecule being a three dimensional chemical structure other than a polynucleotide that binds to the nucleic acid ligand through a mechanism that predominantly depends on Watson/Crick base pairing or triple helix binding, wherein the nucleic acid ligand is not a nucleic acid having the known physiological function of being bound by the target molecule. Nucleic acid ligands include nucleic acids that are identified from a candidate mixture of nucleic acids, said nucleic acid ligand being a ligand of a given target, by the method comprising: a) contacting the candidate mixture with the target, wherein nucleic acids having an increased affinity to the target relative to the candidate mixture may be partitioned from the remainder of the candidate mixture; b) partitioning the increased affinity nucleic acids from the remainder of the candidate mixture; and c) amplifying the increased affinity nucleic acids to yield a ligand-enriched mixture of nucleic acids, whereby nucleic acid ligands of the target molecule are identified. This process, termed the SELEX process, is described in U.S. patent application Ser. No. 07/536,428, filed Jun. 11, 1990, entitled “Systematic Evolution of Ligands by EXponential Enrichment,” now abandoned, U.S. Pat. No. 5,475,096 entitled “Nucleic Acid Ligands”, and U.S. Pat. No. 5,270,163 (see also WO 91/19813) entitled “Nucleic Acid Ligands” each of which is specifically incorporated by reference herein.

One particularly important embodiment of the SELEX process is described in U.S. patent application Ser. No. 08/123,935, filed Sep. 17, 1993, and U.S. patent application Ser. No. 08/443,959 filed May 18, 1995, both entitled “Photoselection of Nucleic Acid Ligands,” and both now abandoned, and U.S. Pat. Nos. 5,763,177, 6,001,577, WO 95/08003, U.S. Pat. No. 6,291,184, U.S. Pat. No. 6,458,539, and U.S. patent application Ser. No. 09/723,718, filed Nov. 28, 2000, each of which is entitled “Systematic Evolution of Nucleic Acid Ligands by Exponential Enrichment: Photoselection of Nucleic Acid Ligands and Solution SELEX,” and each of which describe a SELEX process-based method for selecting nucleic acid ligands containing photoreactive groups capable of binding and/or photocrosslinking to and/or photoinactivating a target molecule. The resulting nucleic acid ligands are referred to interchangeably as “photocrosslinking nucleic acid ligands” and “photoaptamers.”

Automated methods and apparatus for the generation of nucleic acid ligands, including photocrosslinking nucleic acid ligands, are provided in U.S. patent application Ser. No. 09/993,294, filed Nov. 21, 2001, U.S. patent application Ser. No. 09/815,171, filed Mar. 22, 2001, U.S. patent application Ser. No. 09/616,284, filed Jul. 14, 2000, U.S. patent application Ser. No. 09/356,233, filed Jul. 16, 1999, and U.S. Pat. No. 6,569,620, each of which is entitled “Method and Apparatus for the Automated Generation of Nucleic Acid Ligands.”

Photocrosslinking nucleic acid ligands produced by the photoSELEX process have particular utility as capture reagents in multiplexed diagnostic or prognostic medical assays. In one such embodiment, photocrosslinking nucleic acid ligands of targets implicated in disease are attached to a planar solid support in an array format, and the solid support is then contacted with a biological fluid to be analyzed for the presence or absence of the targets. The photocrosslinking nucleic acid ligands are photoactivated and the solid support is washed under very stringent, aggressive conditions (preferably under conditions that denature nucleic acids and/or proteins) in order to remove all non-specifically bound molecules. Bound target is not removed because it is covalently crosslinked to nucleic acid ligand via the photoreactive group. Protein targets bound by the photocrosslinking nucleic acids may then be detected using a reagent or reagents that labels proteins and not nucleic acids with a detectable moiety. Such reagent(s) are referred to as Universal Protein Stains (“UPS”) and are described in PCT/US03/04142, filed Feb. 10, 2003 entitled “Methods for the Multiplexed Evaluation of Photocrosslinking Nucleic Acid Ligands.” The ability to photocrosslink, followed by stringent washing, allows diagnostic and prognostic assays of unparalleled sensitivity and specificity to be performed. Arrays (also commonly referred to as “biochips” or “microarrays”) of nucleic acid ligands, including photocrosslinking nucleic acid ligands and aptamers, and methods for their manufacture and use, are described in U.S. Pat. No. 6,242,246, U.S. patent application Ser. No. 09/211,680, filed Dec. 14, 1998, now abandoned, WO 99/31275, U.S. Pat. No. 6,544,776, U.S. Pat. No. 6,503,715, and U.S. Pat. No. 6,458,543, each of which is entitled “Nucleic Acid Ligand Diagnostic Biochip.” These patents and patent applications are referred to collectively as “the biochip applications,” and are each specifically incorporated herein by reference in their entirety.

Note that throughout this application, various publications and patent applications are mentioned; each is incorporated by reference to the same extent as if each was specifically and individually incorporated by reference.

Adjusting the Quantification Range of Individual Analytes in a Multiplexed Assay

The optimal performance of an analytical assay occurs in the center of the limits of quantification, hereinafter referred to as “LOQ”. The LOQ are the lowest and highest concentration of analyte that can be measured in a sample with acceptable accuracy and precision. The highest concentration should preferably not exceed a loss in accuracy of 10% due to deviation from linearity near saturation. The LOQ should preferably be commensurate with physiological levels of interest. The analyte concentration at which assay saturation occurs is due to a combination of characteristics, most importantly the concentration of capture reagent and its affinity for the analyte.

Capture reagent concentrations for microarrays that measure protein analytes are typically quite low due to the micron scale of the features comprised of capture reagents. Typical microarray capture reagent densities are ˜10⁴ molecules/μm² with feature areas ˜10⁴ μm². Replicate features, say 4 or 5, in a 100 μL sample, therefore, yields a total concentration of capture molecules, Cₜ of:

$${C}_{t} = \frac{4 features \times {10}^{4} molecules \text{/} {µm}^{2} \times {10}^{4} {µm}^{2} \text{/} feature}{6 \times {10}^{23} molecules \text{/} mole \times {10}^{- 4} L} \approx {10}^{- 11} M$$

Surfaces comprised of hydrogel layers, adding a third dimension to the flat surface, can increase this concentration ten-fold. For high affinity capture reagents, such as nucleic acid ligands and antibodies, with K_{d} s better than 1 nM, it is this relatively low concentration of capture reagent that sets the upper limit of quantification. Near saturation, the capture reagent concentration is much less than both the K_{d} and the analyte concentration [A], so the fraction of capture reagents occupied by bound analyte at equilibrium is given by:

$$\frac{[ A : C ]}{{C}_{t}} = \frac{[ A ]}{{K}_{d} + [ A ]} \qquad ( 1 )$$

For a nM or better K_{d}, the upper LOQ will be approximately 5×K_{d} or 5×Cₜ, whichever is larger. Clearly, for protein analytes exceeding nM concentrations (after appropriate dilution), the capture reagents will be saturated and accurate quantification is not possible.

The present invention provides a method for increasing Cₜ for certain capture reagents thereby moving their standard curve—and hence the saturation point—to higher concentration levels for the specific analyte for which Cₜ has been increased. The method involves adding free capture reagent to the solution containing the analyte to be measured. For example, the free capture reagent may be added to the diluent that is used to dilute a biological fluid suspected of containing the analytes to be measured before application of the biological fluid to the surface of a microarray (which microarray comprises the same capture reagent attached thereto). The addition of free capture reagent in solution quantitatively titrates the amount of analyte captured in the assay, lowering saturating levels of analyte to quantifiable levels.

The magnitude of the standard curve shift (and hence the saturation point shift) depends upon the amount of capture reagent immobilized (assumed to be the same here for all capture reagents), the affinity of the capture reagent-analyte pair, and the amount of free capture reagent in the solution containing the analyte. Strict assay linearity will hold at analyte concentrations that are well below the concentration of capture reagent. Mathematically, this is easily seen starting with the equilibrium binding equation for the capture of analyte from solution, A+C⇄A: C,

$${K}_{d} = \frac{[ A ] [ C ]}{[ A : C ]} \qquad ( 2 )$$

where [C] and [A] are the concentration of uncomplexed capture reagent and analyte and [A:C] is the concentration of analyte bound to capture reagent. The mass balance equations for this system are
   Aₜ=[A]+[A:C] (3a)
   Cₜ=[C]+[A:C] (3b)
where Aₜ and Cₜ are the total concentrations, both bound and unbound, of analyte and capture reagent in the system. Cₜ is comprised of both surface immobilized and free soluble capture reagent, whose total concentrations are denoted Cₛ and C_{f}, that is, Cₜ=Cₛ+C_{f}. The assay linearity condition, Aₜ<<Cₜ, allows one to equate [C]≈Cₜ since only a small number of capture reagents will bind analyte under these conditions. Using mass balance (eq. 3), the linearity condition and the equilibrium constant (eq. 2) yields the following expression for the concentration of capture reagent, both surface immobilized and free, bound to analyte

$$[ A : C ] = \frac{{A}_{t} {C}_{t}}{{K}_{d} + {C}_{t}} \qquad ( 4 )$$

Provided the affinity of the complex on the surface is the same as in solution, the concentration of immobilized capture reagent with bound analyte is simply the ratio of surface capture molecules to the total capture molecules times the concentration of complex formed displayed in eq. 4,

$$[ A : {C}_{s} ] = ( \frac{[ {C}_{c} ]}{[ {C}_{t} ]} ) \frac{{A}_{t} {C}_{t}}{{K}_{d} + {C}_{t}} ≡ \frac{{A}_{t} {C}_{s}}{{K}_{d} + {C}_{t}} \qquad ( 5 )$$

Equation 5 can be easily rearranged to give the fraction of surface immobilized capture reagent bound to analyte,

$$\frac{[ A : {C}_{s} ]}{{C}_{s}} ≡ \frac{{A}_{t}}{{K}_{d} + {C}_{s} + {C}_{f}} \qquad ( 6 )$$

The total analyte concentration in a sample that gives rise to particular fraction of bound capture reagents

$${f}_{B} ≡ \frac{[ A : {C}_{s} ]}{{C}_{s}} ,$$

linearly depends on K_{d}, Cₛ and C_{f}, and is given by
Aₜ(C_{f})=f_{B}(K_{d}+Cₛ+C_{f} (7)

The notation Aₜ(C_{f}) emphasizes the fact that, for a fixed K_{d} and Cₛ, in order to obtain a particular f_{B} in the presence of free capture reagent, the amount of total analyte must increase by an amount directly proportional to the concentration of free capture reagent. Since K_{d} and Cₛ are essential characteristics of the capture reagent and the microarray, the two limits of eq. 7 are of interest i.e., where K_{d}>Cₛ and Cₛ>K_{d}.

When K_{d}>Cₛ, eq. 7 reduces to Aₜ(C_{f})=f_{B}(K_{d}+C_{f}) and the ratio of total analyte required to give the same response in the assay to that required with no free capture reagent is simply

$$\frac{{A}_{t} ( {C}_{f} )}{{A}_{t} ( 0 )} = \frac{{K}_{d} + {C}_{f}}{{K}_{d}} \qquad ( 8 )$$

Concentrations of free capture reagent that are less than or equal to K_{d} result in little noticeable shift in the assay response. Free capture reagent present in the assay at a concentration of 10×K_{d} results in an approximately 10-fold shift in the standard curve. Similarly, for capture reagents with K_{d}<Cₛ, the ratio of analyte required to give an equivalent response to the assay with no free capture reagent is

$$\frac{{A}_{t} ( {C}_{f} )}{{A}_{t} ( 0 )} = \frac{{C}_{s} + {C}_{f}}{{C}_{s}} \qquad ( 9 )$$

Concentrations of free capture reagent that are less than or equal to Cₛ result in little noticeable shift in the assay response. Free capture molecules at a concentration of 10×Cₛ results in an approximately 10-fold shift in the standard curve. Determining the concentration of free capture reagent required to shift the concentration range of a given analyte such that it approximately coincides with the center of the LOQ constitutes mere routine experimentation for one skilled in the art guided by eqs 8 and 9.

The presence of free capture reagent for a given analyte does not affect the quantification of any other analyte in the assay. Therefore, it is possible to quantitatively shift the concentration of all highly abundant analytes in a multiplexed assay towards the center of the LOQ while retaining the sensitivity desired for low abundance analytes that would undoubtedly suffer by sample dilution. Thus, the method can be easily applied to multiplexed assays in which thousands of highly abundant analytes—as well as lower abundance analytes—must be simultaneously measured.

Optimal tuning of multiplexed assays according to the methods provided herein also allows for accurate measurements of both up and down regulated analytes that occur under certain non-standard conditions, including, but not limited to, disease states and reactions to medical treatment.

In some embodiments, in addition to altering the quantification range of individual analytes in the multiplexed microarray assay, free capture reagent can be used to reduce nonspecific binding of a cognate analyte to noncognate capture reagent(s). Since the specific interaction of an analyte with its cognate capture reagent in solution will be greater than its affinity for noncognate capture reagents on the array, the effective concentration of free analyte is dramatically reduced, leading to less nonspecific binding by this particular analyte. Therefore, in some embodiments, free capture reagents are added to the diluent for the biological fluid in order to bind and keep in solution those analytes that may be problematic for nonspecific interactions with other non-cognate capture reagents on the array.

The methods provided herein may be used with any capture reagent. Suitable capture reagents include, but are not limited to, antibodies (including fragments thereof), antigens, receptors, proteins, peptides, nucleic acid ligands (including photocrosslinking nucleic acid ligands), and nucleic acid-protein fusions (as described in U.S. Pat. No. 6,537,749, incorporated herein by reference in its entirety). In addition, the methods provided herein are not limited to use with microarrays, but can be used in any multiplexed assay in which capture reagents are associated with solid supports. For example, the methods provided herein may be used with bead-based flow cytometric assays as described in U.S. Pat. No. 6,449,562, incorporated herein by reference in its entirety.

The present invention also provides kits comprising a microarray of capture reagents for the multiplexed detection of a plurality of analytes found in a biological fluid. At least one of the capture reagents on the microarray binds to an analyte that is present in the biological fluid at a concentration that is higher than the upper LOQ for that particular capture reagent. The kit also comprises a container comprising free capture reagent corresponding to at least one capture reagent on the microarray that binds to the abundant analyte. The kit may also comprise one or more containers of buffers or diluents that may be mixed with the biological fluid, along with the free capture reagent, prior to beginning the multiplexed assay. In its simplest embodiment, the kit may include the free capture reagent as part of the diluent to which the biological fluid is added.

### EXAMPLES

The following examples are provided for illustrative purposes only and are not intended to limit the scope of the invention. In particular, it is to be understood that the use of nucleic acid ligands as the capture reagent and proteins as the analytes in the following examples does not limit the nature of capture reagent or analyte that may be used with the general methods of the invention.

### Example 1

### Shifting the Standard Curve of a Single Analyte in a Multiplexed Assay

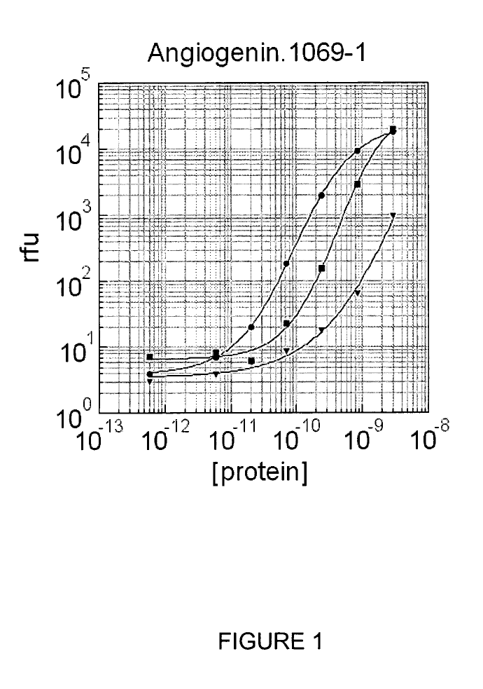

The effect of introducing a free capture reagent into the solution containing the analytes to be measured can be illustrated by examining standard curves in buffer with and without free capture molecules. A microarray on a hydrogel surface that measures 25 protein analytes with 33 distinct aptamers (some analytes are measured with multiple aptamers) was synthesized according to the methods provided in the biochip applications. Twenty-five proteins were serially diluted in buffer, without and with free aptamer to angiogenin (1069-1 having a K_{d} of 20 μM) and applied to separate microarrays to simultaneously generate twenty-five standard curves. Without free angiogenin aptamer, the upper limit of quantification for angiogenin is ˜1 nM, a log above the estimated Cₜ with no free aptamer in solution. Adding 1 and 10 nM free 1069-1 to the diluent shifts the standard curve for angiogenin by ˜0.75 and 1.5 logs to higher concentrations. See FIG. 1. Only the angiogenin standard curve was shifted; none of the other 24 analytes were affected by the addition of 1069-1 to the diluent. In addition to generating the standard curves, angiogenin levels were measured in two serum samples at a 20% dilution with 0, 1 nM and 10 nM free 1069-1. The background subtracted Relative Fluorescence Units (RFU) values for the two samples are summarized in Table 1. The effect of the free aptamer added to the diluent is to reduce the signal on aptamer 1069-1 on the array as the free aptamer concentration increases.

TABLE 1
Angiogenin measured in 20% serum for 0, 1 nM and
10 nM free aptamer

| Free aptamer (nM) | Sample 1 (RFU) | Sample 2 (RFU) |
|---|---|---|
| 0 | 21830 | 13550 |
| 1 | 20269 | 11872 |
| 10 | 9420 | 7979 |

This parallels the observed shift in standard curve to higher concentrations. The same sample concentration will signal lower in an assay with free aptamer present since a proportionate fraction of analyte is distributed between aptamers on the microarray and free in solution.

### Example 2

### Simultaneously Shifting the Standard Curves of a Plurality of Analytes in a Multiplexed Assay

Using the same 25-protein microarray as in Example 1, seven individual aptamers were added to the diluent for sample incubation. See Table 2.

TABLE 2
Seven aptamers added to the sample incubation diluent along
with the affinity for their target analyte and the K_{d}.

| protein analyte | aptamer | [free aptamer] (nM) | K_{d} (nM) |
|---|---|---|---|
| angiogenin | 1069-1 | 30 | 0.02 |
| endostatin | 334-46 | 15 | 0.5 |
| IgE | 869-47 | 2 | 0.1 |
| lactoferrin | 996-35 | 5 | 1.0 |
| L-selectin | 1054-5 | 30 | 4.0 |
| P-selectin | 884-34 | 0.1 | 0.002 |
| TIMP-1 | 905-36 | 2 | 0.15 |

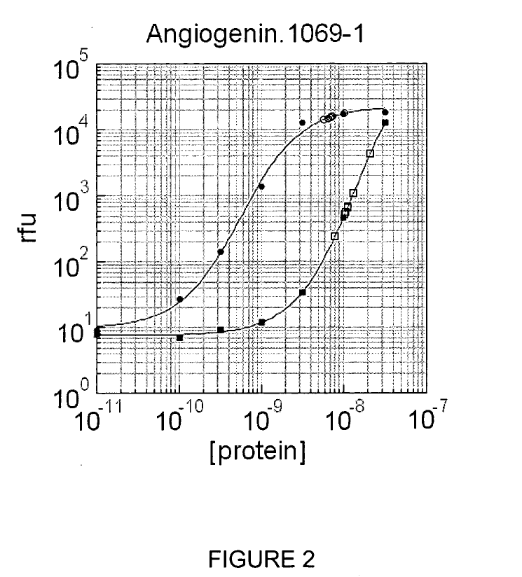

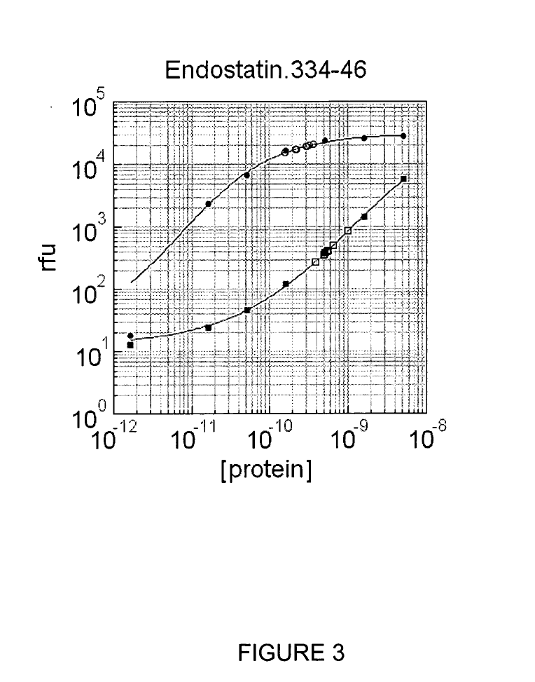

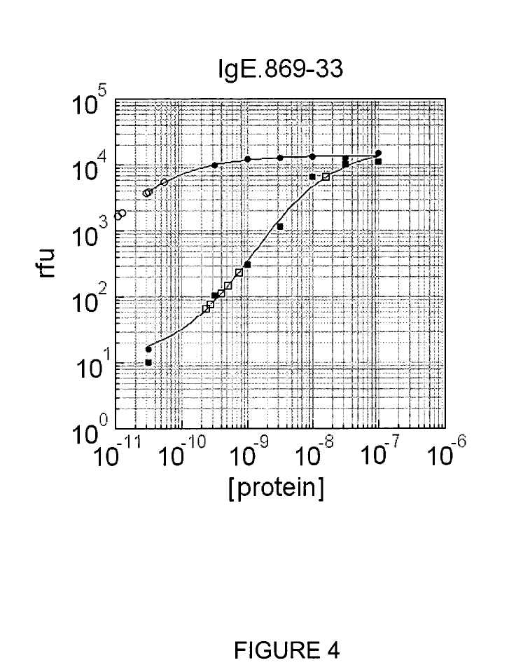

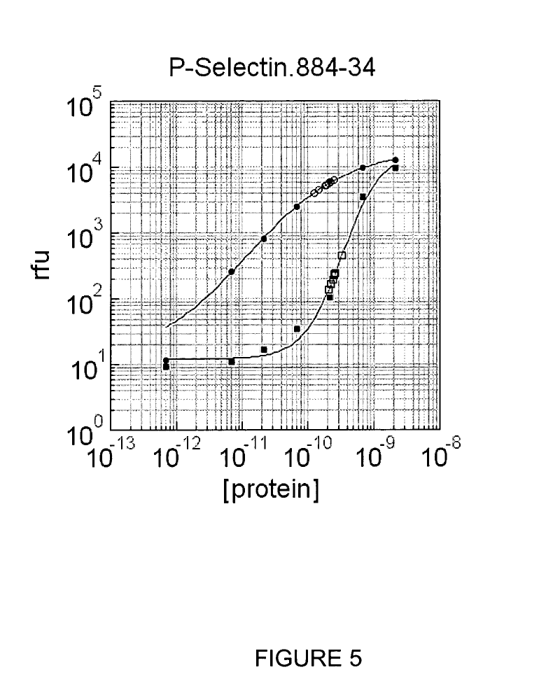

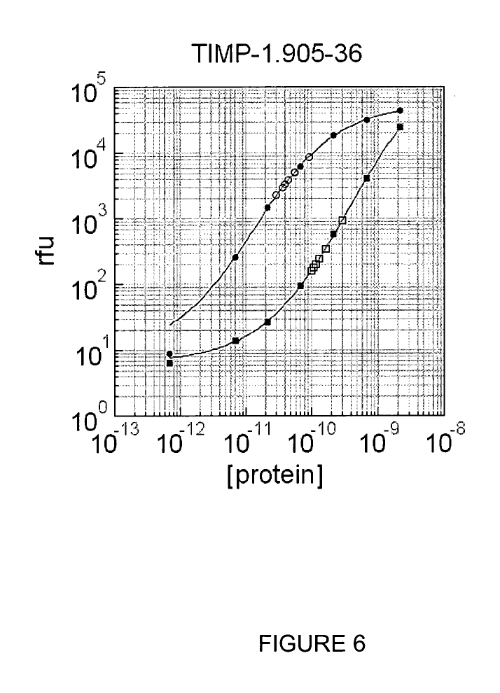

Along with standard curve generation, seven serum samples were run with and without free aptamer. The standard curves for these protein analytes, as well as the serum sample responses are displayed in FIGS. 2-6. The magnitude of the standard curve shift depends upon the amount of aptamer immobilized (assumed to be the same here for all aptamers), the affinity of the aptamer-analyte pair, and the amount of free aptamer in the diluent. The smallest shift is seen for lactoferrin which is less than a tenth of a log while both endostatin and IgE were moved over 2.5 logs from their initial buffer response. Free aptamer that is close to the K_{d} results in little noticeable shift. For aptamers with K_{d}>Cₜ, a 10-fold concentration of free aptamer above the K_{d} results in a 10-fold shift in the standard curves, see results for endostatin (FIG. 3) and TIMP-1 (FIG. 6). For aptamers with K_{d}<Cₜ, a 10-fold concentration of free aptamer above Cₜ results in a 10-fold shift in the standard curve, see results for P-selectin (FIG. 5). Due to the uncertainty in the measured K_{d} values, performed in solution, as well as the uncertainty of surface effects on binding affinities, the results in Table 2 are in reasonable accord with theory.

No standard curve for the other 18 analytes measured with the microarray was affected by the presence of the free aptamer in the protein incubation diluent. Also, the desired effect of lowering the sample responses in serum was observed; all measurements for analytes with free capture molecules are lower compared to those run in buffer alone, see FIGS. 2-6. A direct quantitative comparison of serum measurements with and without free aptamer must be made with care since the measurement without is usually outside of the LOQ. Nevertheless, most calculation agree to within a factor of 2-4. The values determined with the free aptamer present during incubation are presumably more reliable since they are well within the LOQs (which is the purpose of adding the free aptamer).

### Example 3

### Analysis of Possible Matrix Effects

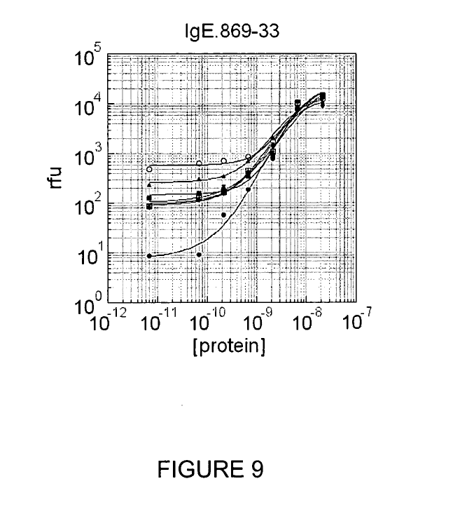

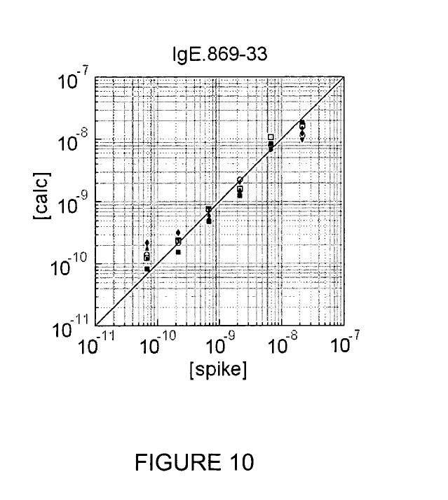

There is a possibility that the presence of free aptamers in Example 1 and 2 introduced matrix effects that could have resulted in a sample bias. For example, different serum samples may have different amounts of material that bind to the free aptamers, reducing their effect in a sample dependent fashion. To address such concerns, a series of serum measurements with spiked samples was performed. If there were large matrix effects, different serum samples would be expected to give rise to different magnitudes of shifts in the spiked samples. This behavior was not observed. The spiked curves all tended to converge on the buffer standard curve at high enough spike levels over the endogenous ones. This is illustrated in FIG. 9 and FIG. 10. The same protein concentrations used to generate the standard curves was used here only spiked into six different serum samples. Data for all 25 spiked analytes were simultaneously generated in the presence of the seven free aptamers in the diluent.

The low protein spike levels are masked by the large endogenous protein levels for both IgE and TIMP-1. As the spiked concentration is increased, the six serum samples tended to converge on the standard curves generated in buffer. Subtracting the endogenous protein concentration, computed from the no protein spike sample, allows for a remarkable recovery of spiked proteins. The computed values all lie near the spiked values. No evidence of individual sample bias was present in these experiments and good analytic behavior was observed. Neither the curves nor the calculated recovery values were different for the analytes with free aptamers compared to those without.

### Example 4

### Reproducibility of Multiplexed Assays with Free Capture Reagent

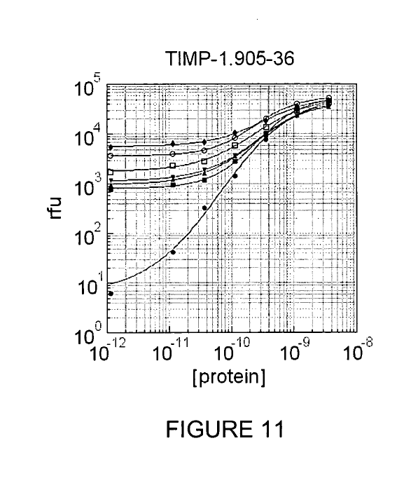

Six serum samples were measured seven times at a 20% dilution. The measurement coefficient of variation, (CV: the standard deviation divided by the mean concentration computed from the seven replicates), was determined for each analyte present. These data are displayed in FIG. 11 as boxplot statistics and show quite reasonable CVs for all analytes in the multiplexed array, with the exception of a noisy VEGF aptamer, 467-65. There is no appreciable difference in the CVs for measurements of analytes with soluble aptamer versus those without. Seventeen aptamers give median CVs of 10% or lower, the remaining are between 10-20%. The overall average median CV is 9.8%, quite an acceptable level of variation for multiple measurements in complex media.

1. A method for decreasing the amount of a first analyte in a biological fluid that is capable of binding to a first capture reagent immobilized on a solid support without decreasing the amount of a second analyte in said biological fluid that is capable of binding to a second capture reagent immobilized on said solid support, the method comprising contacting said biological fluid with said first capture reagent free in solution.

2. The method of claim 1 wherein said first capture reagent is an antibody.

3. The method of claim 1 wherein said first capture reagent is a nucleic acid ligand.

4. The method of claim 1 wherein said first analyte is a protein.

5. The method of claim 1 wherein the dissociation constant, K_{d}, of said first analyte for said first capture reagent is greater than the concentration, Cₛ, of said first capture reagent immobilized on said solid support, and wherein the concentration of said first capture reagent free in solution is greater than said dissociation constant.

6. The method of claim 5 wherein the concentration of said first capture reagent free in solution is about ten-fold greater than said dissociation constant.

7. The method of claim 1 wherein the dissociation constant, K_{d}, of said first analyte for said first capture reagent is less than the concentration, Cₛ, of said first capture reagent immobilized on said solid support, and wherein the concentration of said first capture reagent free in solution is greater than Cₛ.

8. The method of claim 7 wherein the concentration of said first capture reagent free in solution is about ten-fold greater than Cₛ.

9. A method for increasing the saturation point for an analyte of a capture reagent immobilized on a solid support, the method comprising contacting said solid support with said capture reagent free in solution.

10. A method for determining the concentration of an analyte in a biological fluid, the method comprising:
a) providing a first quantity of a capture reagent capable of binding to said analyte, wherein said first quantity of said capture reagent is immobilized on a solid support;
b) contacting said solid support with a mixture comprising said biological fluid and a second quantity of said capture reagent;
c) measuring the amount of said analyte bound to said first quantity of capture reagent; and
d) calculating the concentration of said analyte in said biological fluid based on the measurement made in step c), the concentration of said second quantity of said capture reagent in the mixture of step b), and the K_{d} of said capture reagent.

11. A method for lowering the nonspecific binding of an analyte in a biological fluid to a non-cognate capture reagent immobilized on a solid support, the method comprising contacting said biological fluid with a capture reagent capable of specifically binding to said analyte, wherein said capture reagent capable of specifically binding to said analyte is free in solution in said biological fluid.

12. A method for increasing the effective concentration of a capture reagent immobilized on a solid support, the method comprising contacting said solid support with said capture reagent free in solution.
